(19) United States
(12) Patent Application Publication
(10) Pub. No.: US 2006/0147479 A1
(43) Pub. Date: Jul. 6, 2006

(54) Angiogenic medical cyanoacrylate adhesive

(76) Inventors: Neil Joseph Atkin, Beverley (GB); Chritopher Ansell, North Yorkshire (GB); Andrew Patrick Jackson, York (GB)

Correspondence Address: CHIEF PATENT COUNSEL;SMITH & NEPHEW, INC., 1450 BROOKS ROAD, MEMPHIS, TN, 38116, US

(21) Appl. No.: 10/535,240

(22) PCT Filed: Nov. 17, 2003
(86) PCT No.: PCT/GB03/04965

§ 371 Date: Dec. 21, 2005

(30) Foreign Application Priority Data
Nov. 20, 2002 (GB) 0227043.7

(51) Int. Cl. A61L 24/00

(52) U.S. Cl. 424/400

(57) ABSTRACT

Tissue adhesive comprising a cyanoacrylate in combination with an angiogenic factor such as butyric acid or a derivative or a precursor thereof.

The invention relates to adhesives and angiogenesis, in particular the use of novel angiogenic adhesives in surgery.

Adhesion of tissue is an integral part of all surgical procedures, including closure of skin wounds, reconstruction of nerve ruptures, re-attachment of transplanted tissue, sealing of blood vessels, treatment of pneumothorax and fistulas, support of vascular and intestinal anastomoses, treatment of chondral- and osteochondral defects, fracture healing, treatment of meniscal tears and ruptured ligaments, repair of tendon damage or muscle damage and attachment of implanted biomaterials and tissue engineered devices.

The fundamental aim of all tissue adhesives is to hold tissue together for long enough to allow a natural biological repair. Biological repair typically involves the activation of cells in the tissue to a repair mode and, critically, the stimulation of angiogenesis to provide repair cells, nutrition and oxygen to the activated cells.

There is extensive literature describing the use of bioactives to stimulate tissue repair. However, stimulation of tissue repair mechanisms alone, such as angiogenesis, using biological actives will not be enough to heal a large defect between tissues. It is essential that the two tissues to be integrated are held in close apposition at the macroscopic level in order to allow the biological mechanisms to bond the tissue at the microscopic level.

To date tissue adhesion is mainly done using mechanical fastening techniques such as suturing or stapling. There are occasions, however, when the application of a biological glue or adhesive would be beneficial. For example, sutures are inappropriate for cartilage repair as these cause non-healing defects to form in the cartilage where they are placed. Suturing of internal tissue and organs is also slow and technically difficult compared to application of an adhesive. Other tissues that may require adhesion such as bone or certain implants may be too hard for sutures or staples whilst other soft tissues may be too fragile for the suture or staple to hold under tension.

Consequently, adhesives have been developed for biological applications, including biological adhesives such as fibrin and synthetic adhesives such as cyanoacrylates.

Biological adhesives that utilise naturally occurring adhesive processes such as the blood coagulation cascade (fibrin) have a number of advantages. They are readily accepted by the body and break down completely to allow a full biological repair. However, the bonding strength of such adhesives is well below the levels required for many applications, including all those where the bonded tissue is under any significant tension.

A number of synthetic adhesives have been manufactured for industrial and consumer use. Some of these, including cyanoacrylates, have been used to glue biological tissues.

The advantage of using cyanoacrylates is that they form an extremely strong bond between tissues. However, they have not replaced the use of other fixation devices because the cyanoacrylate acts as a barrier to biological repair.

It would therefore be desirable to produce a cyanoacrylate adhesive that actively stimulated tissue repair.

It is a well established fact that, in most tissues, stimulation of angiogenesis results in the acceleration of tissue repair whilst inhibition of angiogenesis can result in ischaemia and tissue death.

There is published literature on the delivery of angiogenic factors to stimulate tissue repair. WO 97/16176, WO 01/03607 and U.S. Pat. No. 6,152,141 describe the release of angiogenic facors to accelerate blood vessel repair, EP 0,295,721 describes the promotion of meniscal healing with angiogenic factors, whilst EP 0,530,804 describes the use of angiogenic materials to promote cartilage and bone healing. However, none of these prior art teach the incorporation of angiogenic factors into cyanoacrylate adhesives.

Butyric acid is a potent angiogenic agent and has been used as an angiogenic factor for the treatment of burns, wounds and bone fractures. Butryic acid, also known as butanoic acid, is a four carbon fatty acid. The literature suggest that the local release of 10-1000 ng of butyric acid is sufficient to achieve the desired angiogenic effect. However, butyric acid is known to be removed rapidly from the body and therefore for therapeutic angiogenic applications it has been suggested that it be incorporated into a sustained release delivery vehicle. A lipid angiogenic factor has been isolated from omentum (Catsimpoolas et al., 1984, JAMA 252:2034-2036). The angiogenic factor was found to be monobutyrin (Wilkinson et al., 1991, J. Biol. Chem. 266:16886-16891).

Monobutyrin can be considered to be a prodrug of butyric acid. Other prodrugs include tributyrin. Tributyrin can be hydrolysed to release butyric acid (Chen et al, 1994, Cancer Research 54, 3494-3499, Bohmig et al, 1999, Transplant Immunology, 7, 221-227). Tributyrin has been proposed for use in anti-cancer therapies where it is desirable to inhibit angiogenesis, it has not been considered has an angiogenic drug.

There is prior art describing the use of cyanoacrylate adhesives in tissue repair applications (Barley et al., U.S. Pat. No. 6,342,213, methods for treating non-suturable wounds by use of cyanoacrylate adhesives; Hyon et al., U.S. Pat. No. 6,316,523, adhesive composition for surgical use; Shalaby, U.S. Pat. No. 6,299,631, polyester/cyanoacrylate tissue adhesive formulations; Kotzev, U.S. Pat. No. 6,224,622, bioabsorable cyanoacrylate tissue adhesives). However, none of these patents disclose the addition of an angiogenic component to the cyanoacrylate.

There is literature describing the incorporation of active molecules into tissue sealants (MacPhee et al., U.S. Pat. No. 6,117,425, Supplemented and unsupplemented tissue sealants, method of their production and use), but this patent makes no mention of cyanoacrylates, despite providing long lists of other frequently used tissue sealants.

Simple active molecules such as Iodine have been incorporated into cyanoacrylates (Askill et al., U.S. Pat. No. 6,214,332, methods for closing suturable wounds by use of cyanoacrylate ester compositions comprising an antimicrobial agent), but angiogenic agents are usually proteins or chemically active nucleophiles that will cure the cyanoacrylate prematurely, rendering it useless as a tissue adhesive. Thus there is prejudice in the art that suggests that it would not be possible to incorporate an angiogenic agent into a cyanoacrylate.

It is an objective of the present invention to provide a cyanoacrylate adhesive for use in biological applications that releases a biologically active angiogenic agent.

We have discovered that tributyrin and some related molecules (butyric acid prodrugs), which are also capable of stimulating angiogenesis, surprisingly do not cause cyanoacrylate to prematurely cure.

Accordingly, to the present invention there is provided a tissue adhesive comprising a cyanoacrylate in combination with an angiogenic factor, which is releasable in amounts that will cause a pharmacological effect.

The cyanoacrylate adhesive will typically be selected from the group consisting of alkyl 2-cyanoacrylate, alkenyl 2-cyanoacrylate, alkoxyalkyl 2-cyanoacrylate, and carbalkoxyalkyl 2-cyanoacrylate, wherein the alkyl group of said one or more cyanoacrylates has 1 to 16 carbon atoms.

The cyanoacrylate will preferably be selected from the group consisting of methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate.

In a first embodiment of this invention the angiogenic factor is butyric acid or a derivative or precursor thereof.

The angiogenic factors may include:
Butyric acid (butanoic acid, C₄H₈O₂) and butyric acid salts, including sodium, potassium, calcium, ammonium and lithium salts
Butyric acid derivatives and polymers containing butyric acid residues
α-monobutyrin (1-glycerol butyrate; 1-(2,3 dihydroxypropylbutanoate; C₇H₁₄O₄)
α-dibutyrin (1,3-glyceroldibutyrate; 1,3-(2 hydroxypropyl)dibutanoate; C₁₁H₂₀O₅)
β-dibutyrin (1,3-glyceroldibutyrate; 1,2-(3 hydroxypropyl)dibutanoate; C₁₁H₂₀O₅)
tributyrin (glcerol tributyrate; 1,2,3-(propyl)tributanoate; C₁₅H₂₆O₆)
hydroxybutyrate and polymers containing hydroxybutyric acid residues

The angiogenic factor is added to the cyanoacrylate in such proportions as to result in an adhesive strength, aptly of not less than 0.05 Mpa, preferably at least 0.2 MPa and more preferably at least 0.5 Mpa. Typically the resultant adhesive strength should range from 0.05 to 0.8 Mpa.

Once cured, the cyanoacrylate will aptly release at least 1 ng/ml adhesive of the angiogenic factor. Suitably the cyanoacrylate will release less than 100 μg of angiogenic factor, although preferably it will release less than 10 μg and more preferably less than 1 μg of the angiogenic factor.

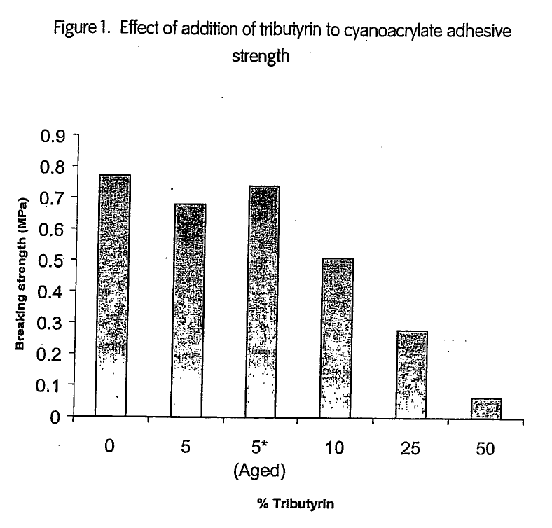

The invention will be illustrated by references to the following examples and the accompanying drawings in which FIG. 1 shows that 5% to 50% (w/w) tributyrin can be added to cyanoacrylate without an unacceptable loss of adhesive property.

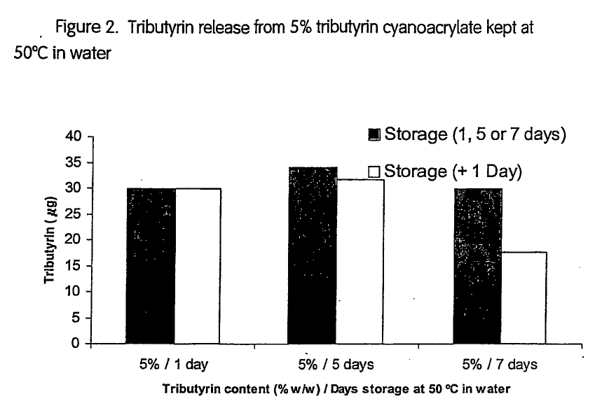

FIG. 2 shows that tributyrin is released from a 5% w/w tributyrin cyanoacrylate sample over a 7 day period.

### EXAMPLE 1

0.5 g of the sterile tributyrin was added to cyanoacrylate (9.5 g) to produce a 5% (w/w) blend in a sterile plastic universal tube. This was mixed for 2 minutes to ensure a homogenous blend. 40 μl aliquots of the blended adhesive were applied to freshly cut surfaces of costal cartilage. The two surfaces were held together for 1 minute allowing fixation and curing was allowed to continue for an additional hour in aqueous conditions. The bond strength was tested using a Nene MC3000 machine, where the applied force required to separate the bonded pieces of cartilage was recorded. The data showed that 5% to 50% (w/w) tributyrin can be added to cyanoacrylate without an unacceptable loss of adhesive property.

### EXAMPLE 2

0.5g of the sterile tributyrin was added to cyanoacrylate (9.5 g) to produce a 5% (w/w) blend in a sterile plastic universal tube. This was mixed for 2 minutes to ensure a homogenous blend. Small disks of the blended adhesive were cast onto a basic solution (dilute triethylamine 0.1% aq). The disks were removed, washed briefly and dried. The disks were then placed into 2 ml water and stored at 50° C. for 1, 5 or 7 days with continuous agitation. The water was removed then added to 2 mls dichloromethane in order to extract any released tributyrin. The dichloromethane layer was analysed using gas chromatography and the amount of tributyrin recorded. The disks were dried and then placed into fresh water at 50° C. for an additional day. The amount of tributyrin released into the fresh water was measured as described for the first samples. The data showed that tributyrin is released from a 5% w/w tributyrin cyanoacrylate sample over a 7 day period.

1. A tissue adhesive comprising a cyanoacrylate in combination with an angiogenic factor.

2. A tissue adhesive according to claim 1 wherein the angiogenic factor is butyric acid or a derivative or precursor thereof.

3. A tissue adhesive according to claim 2 wherein the angiogenic factor is a butyric acid salt.

4. A tissue adhesive according to claim 3 wherein the angiogenic factor is a sodium, potassium, calcium, ammonium or lithium butyric acid salt.

5. A tissue adhesive according to claim 2 wherein the butyric acid, derivative or precursor thereof is present as a polymer containing residues of butyric acid or hydroxybutyric acid.

6. A tissue adhesive according to claim 1 wherein the angiogenic factor is α-monobutyrin, α-dibutyrin, β-dibutyrin or tributyrin.

7. A tissue adhesive according to claim 1 wherein the angiogenic factor is hydroxybutyrate.

8. A tissue adhesive according to claim 1 wherein the cyanoacrylate is selected from the following group: alkyl 2-cyanoacrylate, alkenyl 2-cyanoacrylate, alkoxyalkyl 2-cyanoacrylate, or carbalkoxyalkyl 2-cyanoacrylate.

9. A tissue adhesive according to claim 8 wherein the alkyl group of said cyanoacrylates has 1 to 16 carbon atoms.

10. A tissue adhesive according to claim 1 wherein the cyanoacrylate is selected from the following group: methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate, and 2-propoxyethyl 2-cyanoacrylate.

11. A tissue adhesive according to claim 1 that has an adhesive strength of at least 0.05 Mpa.

12. A tissue adhesive according to claim 1 that has an adhesive strength of between 0.05 and 0.8 Mpa.

13. A tissue adhesive according to claim 1 that releases at least 1 ng/ml adhesive of the angiogenic factor.

14. A tissue adhesive according to claim 1 that releases at least 10 ng of the angiogenic factor.

15. A tissue adhesive according to claim 1 that releases less than 10 μg of the angiogenic factor.

16. A tissue adhesive according to claim 1 that releases less than 1 μg of the angiogenic factor.

17. A tissue adhesive according to claim 1 that contains at least 5% w/w of angiogenic factor.
